(19) United States
(12) Patent Application Publication
(10) Pub. No.: US 2021/0030680 A1
(43) Pub. Date: Feb. 4, 2021

(54) NANOPARTICLE COMPOSITIONS AND METHODS OF USE OF PARP INHIBITOR FOR TREATMENT OF CANCER

(71) Applicant: THE BOARD OF REGENTS OF THE UNIVERSITY OF TEXAS SYSTEM, Austin, TX (US)

(72) Inventors: Raushan T. KURMASHEVA, San Antonio, TX (US); Kytai T. NGUYEN, Arlington, TX (US)

(73) Assignee: The Board of Regents of University of Texas System, Austin, TX (US)

(21) Appl. No.: 15/733,741

(22) PCT Filed: Apr. 15, 2019
(86) PCT No.: PCT/US2019/027472

§ 371 Date: Oct. 12, 2020

Related U.S. Application Data
(60) Provisional application No. 62/657,292, filed on Apr. 13, 2018.

(51) Int. Cl. A61K 9/14; A61P 35/00; A61K 9/00; A61K 31/5025

(52) U.S. Cl. CPC A61K 9/146; A61P 35/00; B82Y 5/00; A61K 31/5025; A61K 9/0053

(57) ABSTRACT

Described herein are methods of treatment of cancer by administering a therapeutically effective amount of a cytotoxic alkylating agent and a nanoparticle formulation containing an inhibitor of poly (ADP-ribose) polymerase 1. Also disclosed are treatment regimens involving parenteral administration of a therapeutically effective amount of a cytotoxic alkylating agent and a nanoparticle formulation containing an inhibitor of poly (ADP-ribose) polymerase 1.

### CROSS-REFERENCE TO RELATED APPLICATIONS

This application claims priority to and the benefit of U.S. Application No. 62/657,292 filed on Apr. 13, 2018.

### FIELD OF INVENTION

This disclosure relates to compositions and methods of use of inhibitors of poly (ADP-ribose) polymerase (PARP) for the treatment of cancers.

### BACKGROUND

Ewing sarcoma (EwS) is a heterogeneous family of highly malignant, undifferentiated mesenchymal origin tumors that mainly affect children and young adults. Ewing family of sarcomas comprises the fourth most common highly malignant childhood cancer. EwS is defined by a tumor-specific chromosomal translocation. In approximately 85% of all tumors, the EWSR1 gene on chromosome 22 is fused to a member of E26 transformation-specific sequence (ETS) family of transcription factors, the FLI1 gene on chromosome 11. In the remaining 15% of Ewing tumors, the EWSR1 is fused to other members of ETS family, mostly the ERG gene on chromosome 21. DNA damage induced by expression of EWSR1-FLI1 fusion gene is potentiated by poly-ADP ribose polymerase 1 (PARP1) inhibition in Ewing cells, where EWSR1-FLI1 genes act in a positive feedback loop to maintain the expression of PARP1. PARP catalyzes post-translational ADP-ribosylation of nuclear proteins that signal and recruit other proteins to repair damaged DNA and is activated by single-strand DNA breaks. Ewing sarcoma cell lines are hypersensitive to inhibitors of PARP1. Although dose intensification and compression has improved outcome, for patients with advanced or metastatic disease survival remains poor.

### SUMMARY

Disclosed herein are compositions and methods addressing the shortcomings of the art, and may provide any number of additional or alternative advantages, including more effective and less toxic therapy for certain cancers. Embodiments of the compositions and the methods of treatment of cancer in a subject include administering a cytotoxic alkylating agent and a nanoparticle formulation containing an inhibitor of poly (ADP-ribose) polymerase 1 or pharmaceutical acceptable derivatives thereof. An embodiment of the method of treatment of cancer in a subject includes administering a therapeutically effective amount of a cytotoxic alkylating agent and a nanoparticle formulation containing an inhibitor of poly (ADP-ribose) polymerase 1 or pharmaceutical acceptable derivatives thereof. This method can be used to treat cancers, such as Ewing's sarcoma, breast cancer, ovarian cancer, colorectal cancer, prostate cancer, melanoma, and lung cancer. The cytotoxic alkylating agent can be one or more of a nitrogen mustard, a nitrosurea, an ethylenimine, an alkylsulfonate, a hydrazine, a triazines, or a metal salt. The cytotoxic alkylating agent can be one or more of cyclophosphamide, melphalan, ifosfamide, or irinotecan. In an embodiment, the cytotoxic alkylating agent is an imidazotetrazine. The cytotoxic alkylating agent can be temozolomide. The inhibitor of poly (ADP-ribose) polymerase 1 can be a benzimidazole-based compound, including but not limited to niraparib, rucaparib, olaparib, veliparib, or talazoparib. In an embodiment, the nanoparticle formulation contains poly lactic-co-glycolic acid.

Also disclosed here are pharmaceutical compositions for treatment of cancer containing a nanoparticle formulation containing an inhibitor of poly (ADP-ribose) polymerase 1 or a pharmaceutical acceptable derivative thereof. The inhibitor of poly (ADP-ribose) polymerase 1 can be a benzimidazole-based compound, including but not limited to niraparib, rucaparib, olaparib, veliparib, or talazoparib. In an embodiment, the nanoparticle formulation contains poly lactic-co-glycolic acid. In an embodiment, the poly lactic-co-glycolic acid contains a 1:1 ratio of lactic acid and glycolic acid. In an embodiment, the nanoparticle formulation contains high molecular weight poly lactic-co-glycolic acid. This nanoparticle formulation can be prepared for parenteral administration. Also disclosed are dosage regimens involving the parenteral administration of the inhibitor of poly (ADP-ribose) polymerase 1 or a pharmaceutical acceptable derivative thereof and the oral administration of the cytotoxic alkylating agent.

Numerous other aspects, features and benefits of the present disclosure may be made apparent from the following detailed description taken together with the figures. The pharmaceutical compositions can include compounds described herein along with other components, or ingredients depending on desired prevention and treatment goals. It should be further understood that both the foregoing general description and the following detailed description are exemplary and explanatory and are intended to provide further explanation of the invention as claimed.

### BRIEF DESCRIPTION OF THE DRAWINGS

The following figures illustrate some of the embodiments described in the disclosure.

FIG. 1 is a graphical representation of the changes in volume of the tumors over time in animals administered with blank nanoparticles, according to an embodiment.

FIG. 2 is a graphical representation of the changes in volume of the tumors over time in animals administered with temozolomide alone, according to an embodiment.

FIG. 3 is a graphical representation of the changes in volume of the tumors over time in animals that were administered the nanoparticle formulation of talazoparib, according to an embodiment.

FIG. 4 is a graphical representation of the changes in volume of the tumors over time in animals that were administered with temozolomide and the nanoparticle formulation of talazoparib, according to an embodiment.

FIG. 5 is a graphical representation of the relative tumor volume (RTV) over time in animals that were administered with blank nanoparticles, temozolomide alone, nanoparticle formulations of talazoparib, and temozolomide in combination with nanoparticle formulations of talazoparib, corresponding to FIGS. 1- 4 in the present application.

FIGS. 6A-6C are images showing enhanced permeability and retention effect of the nanoparticle formulations of talazoparib in combination with temozolomide.

FIG. 7 is a graphical representation of the changes in the body weight of animals who were administered nanoparticle formulations of talazoparib, temozolomide alone, and nanoparticle formulations of talazoparib in combination with temozolomide, according to an embodiment.

FIG. 8 is a graphical representation of the release profile of talazoparib (TLZ) from nanoparticles of two different molecular weights, according to an embodiment.

### DETAILED DESCRIPTION

Reference will now be made to the exemplary embodiments illustrated in the drawings, and specific language will be used here to describe the same. It will nevertheless be understood that no limitation of the scope of the invention is thereby intended. Alterations and further modifications of the inventive features illustrated here, and additional applications of the principles of the inventions as illustrated here, which would occur to one skilled in the relevant art and having possession of this disclosure, are to be considered within the scope of the invention.

Methods of treatment of cancer in subjects include the administration of a therapeutically effective amount of a cytotoxic alkylating agent and a nanoparticle formulation containing an inhibitor of poly (ADP-ribose) polymerase 1 or pharmaceutical acceptable derivatives thereof. This method can be used to treat cancers, such as Ewing's sarcoma, breast cancer, ovarian cancer, colorectal cancer, prostate cancer, melanoma, and lung cancer. In certain embodiments, the cytotoxic alkylating agent and the nanoparticle formulation can be administered in accordance with different dosage regimens. The dosage regimens can include different schedule of doses of the cytotoxic alkylating agent and the nanoparticle formulation, such that their presence in the subject is within the window of therapeutic efficacy of the cytotoxic alkylating agent and the PARP1 inhibitor. For example, a dosage regimen can include the parenteral administration of the inhibitor of poly (ADP-ribose) polymerase 1 and the oral administration of the cytotoxic alkylating agent. Another example of the dosage regimen is a parenteral dose of a nanoparticle formulation containing talazoparib administered once a week combined with an oral dose of cytotoxic alkylating agent that may be administered once or multiple times per day. The cytotoxic alkylating agents can include one or more of a nitrogen mustard, a nitrosurea, an ethylenimine, an alkylsulfonate, a hydrazine, a triazines, or a metal salt. The cytotoxic alkylating agent can be one or more of cyclophosphamide, melphalan, ifosfamide, or irinotecan. In certain embodiments, the cytotoxic alkylating agent is an imidazotetrazine. In certain embodiments, the cytotoxic alkylating agent that can be used in this method is temozolomide.

Methods of treatment of cancer in subjects include the administration of a therapeutically effective amount of a nanoparticle formulation containing an inhibitor of poly

(ADP-ribose) polymerase 1 (PARP1) or pharmaceutical acceptable derivatives thereof. This method can be used to treat cancers that implicate PARP1, such as Ewing's sarcoma, breast cancer, ovarian cancer, colorectal cancer, prostate cancer, melanoma, and lung cancer. A variety of PARP1 inhibitors can be utilized based on desired potency of the PARP1 trapping to DNA. An example of an inhibitor of poly (ADP-ribose) polymerase 1 that can be used in these methods is talazoparib. Example of inhibitors of poly (ADP-ribose) polymerase 1 that can be used in these methods are other benzimidazole-based compounds, such as niraparib, rucaparib, olaparib, and veliparib. In certain embodiments, the nanoparticle formulation contains one or more inert compounds, such as chitosan, dextran, gelatin, alginates, lipids, starch, polylactic acid, poly(cyano)acrylates, polyethyleinemine, block copolymers, or polycaprolactone. In other embodiments, the nanoparticles may include biodegradable polymers, such as poly lactic-co-glycolic acid (PLGA), poly lactic acid (PLA), poly glycolic acid (PGA), nature polymers like gelatin and chitosan, and lipids, including liposome, or a combination thereof. In certain embodiments, the nanoparticle formulation comprises poly lactic-co-glycolic acid. In some embodiments, the nanoparticles can be coated and/or incorporated with targeting molecules such as ligands, peptides, and antibodies that will bind to the receptors overexpressed on cancer cells. In some embodiments, the nanoparticles encapsulate the PARP1 inhibitor. In some embodiments, the PARP1 inhibitor is incorporated as part of the nanoparticle layer.

Embodiments of pharmaceutical compositions disclosed here include a therapeutically effective amount of nanoparticles containing talazoparib, which are suitable for parenteral administration. In certain embodiments, the nanoparticles contain one or more inert compounds, such as chitosan, dextran, gelatin, alginates, lipids, starch, polylactic acid, poly(cyano)acrylates, polyethyleinemine, block copolymers, or polycaprolactone. In certain embodiments, the nanoparticle formulation further comprises poly lactic-co-glycolic acid. The pharmaceutical compositions for parenteral administration can include aqueous and non- aqueous sterile injection mixtures. The pharmaceutical compositions for parenteral administration may contain one or more of buffers, solvents, antioxidants, preservatives, suspending agents, thickening agents, and solutes, which render the composition suitable for entering the bloodstream of the patient. These pharmaceutical compositions can be packaged in unit-dose or multi-dose containers as fluid compositions. In other embodiments, these pharmaceutical compositions can be packaged as freeze-dried/lyophilized compositions requiring only the addition of the sterile fluid before administration to a patient. In certain embodiments, these pharmaceutical compositions are formulated for intramuscular administration.

Also disclosed here are pharmaceutical compositions that contain a therapeutically effective amount of a cytotoxic alkylating agent and a nanoparticle formulation containing an inhibitor of poly (ADP-ribose) polymerase 1 or pharmaceutical acceptable derivatives thereof. An example of a cytotoxic alkylating agent is temozolomide. An example of an inhibitor of poly (ADP-ribose) polymerase 1 is talazoparib. In an embodiment, the nanoparticle formulation contains poly lactic-co-glycolic acid. Also disclosed here are treatment regimens involving periodic administration of temozolomide and a nanoparticle formulation containing temozolomide.

The pharmaceutical compositions contain a therapeutically effective amount of a cytotoxic alkylating agent together with one or more acceptable carriers therefor and optionally other therapeutic ingredients. The carrier(s) is a compound compatible with the other ingredients of the pharmaceutical composition and not deleterious to the patient. The dosage regimen of the pharmaceutical compositions may include any controlled release dosage form containing a therapeutically effective amount of a cytotoxic alkylating agent. In certain embodiments, the controlled release dosage form is an oral dosage form such as, for example, a tablet or capsule.

As used herein, unless otherwise noted, the terms “treating”, “treatment” and the like, shall include the management and care of a subject for the purpose of combating a disease, condition, or disorder and includes the administration of compositions disclosed here to prevent the onset of certain symptoms or complications of cancer, alleviate the symptoms or complications of cancer, or eliminate the cancer. The term “subject” or “patient” as used herein, refers to an animal, preferably a mammal, most preferably a human, who has been the object of treatment, observation, or experiment. Preferably, the subject has experienced or exhibited at least one symptom of the disease or disorder to be treated or prevented.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes treatment of the patient. A “pharmaceutical composition” refers to a composition containing an active ingredient, such as a PARP1 inhibitor or a cytoxic alkylating agent, or a pharmaceutically acceptable derivative thereof. The pharmaceutical composition can also include at least one pharmaceutically acceptable carrier or excipient. The purpose of the pharmaceutical composition is to facilitate administration of the active ingredients to a subject. In some embodiments, the pharmaceutical composition includes two or more pharmaceutically acceptable carriers and/or excipients. The term “pharmaceutically acceptable derivative” as used herein refers to and includes any pharmaceutically acceptable salt, pro-drug, metabolite, ester, ether, hydrate, polymorph, solvate, complex, and adduct of a compound described herein which, upon administration to a subject, is capable of providing (directly or indirectly) the active ingredient. For example, a pharmaceutically acceptable derivative thereof of a PARP1 inhibitor includes all derivatives of the PARP1 inhibitor (such as salts, pro-drugs, metabolites, esters, ethers, hydrates, polymorphs, solvates, complexes, and adducts) which, upon administration to a subject, are capable of providing (directly or indirectly) the PARP1 inhibitor. As used herein, the term “pharmaceutically acceptable salt” refers to those salts of a compound, which retain the biological effectiveness and properties of the parent compound. And unless otherwise indicated, a pharmaceutically acceptable salt includes salts of acidic or basic groups, which may be present in the compounds disclosed herein. The present disclosure also relates to a process for the preparation of the above pharmaceutically acceptable salts, their derivatives, their analogs, their tautomeric forms, their stereoisomers, their polymorphs, and pharmaceutical compositions containing them.

Illustratively, an effective amount of the compositions disclosed here ranges from nanogram/kg to milligram/kg amounts for pediatric and adult patients. Equivalent dosages for lighter or heavier body weights can readily be determined. The dose should be adjusted to suit the individual to whom the composition is administered and will vary with age, weight and metabolism of the individual. The exact amount of the composition required will vary from subject to subject, depending on the species, age, weight, and general condition of the subject, the particular peptide or polypeptide used, its mode of administration and the like. An appropriate amount can be determined by one of ordinary skill in the art using only routine experimentation given the teachings herein. One skilled in the art will realize that dosages are best optimized by the practicing physician or veterinarian and methods for determining dose amounts and regimens and preparing dosage forms are described, for example, in Remington: The Science and Practice of Pharmacy, 22nd edition.

Optimal dosages to be administered may be readily determined by those skilled in the art, and will vary with the particular composition used, the mode of administration, the strength of the preparation, the mode of administration, the number of consecutive administrations within a limited period of time (e.g. per day or per week) and the advancement of the disease condition. In addition, factors associated with the particular patient being treated, including patient age, weight, diet and time of administration, will result in the need to adjust dosages.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any amount or range therein.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental or measurement conditions or both for such given value.

Ewing sarcoma (EwS) cells are hypersensitive to inhibition of the DNA repair protein PARP. A combination of talazoparib (TLZ) with temozolomide (TMZ) causes regression of ˜50% of EwS xenograft models. Talazoparib is an orally available inhibitor of PARP that selectively binds to PARP and prevents PARP-mediated DNA repair of single strand DNA breaks via the base-excision repair pathway. This enhances the accumulation of DNA strand breaks, promotes genomic instability, and eventually leads to apoptosis. Temozolomide is an orally available cytotoxic alkylating agent. Temozolomide is converted at physiologic pH to the short-lived active compound, monomethyl triazeno imidazole carboxamide (MTIC). The cytotoxicity of MTIC is due primarily to methylation of DNA at the O₆ and N₇ positions of guanine residues of DNA, resulting in inhibition of DNA replication. Temozolomide is metabolized to MITC at all sites. Administration of these single agents had little or no antitumor activity with respect to EwS. However, the combination of talazoparib and temozolomide causes synergistic toxicity and necessitates reducing the dose of temozolomide to ˜13% of its single agent maximum-tolerated dose in animals and a similar dose reduction in children in clinical trials.

Disclosed here are compositions containing nanoparticle formulations of talazoparib (NpTLZ), alone and in combination with temozolomide. The nanoparticle formulations of talazoparib can be administered as injectable compositions. These nanoparticle formulations of talazoparib are superior to talazoparib administered by itself in their ability to predominantly accumulate at the site of the tumors. These nanoparticle formulations increased accumulation, increased the bioavailability, and lowered the systemic toxicity of talazoparib. These nanoparticle formulations increased tumor drug delivery, reduced normal tissue toxicity (mainly thrombocytopenia), and allows escalation of temozolomide dose.

In an embodiment, talazoparib loaded nanoparticles were made using the standard emulsion method. In this method, talazoparib in a powder form and PLGA were added to a polyvinyl alcohol solution and subjected to a sonication procedure. The liquid components in the mixture were partially removed, such as by evaporation. The talazoparib loaded nanoparticles were collected from the resulting suspension, purified, and lyophilized to prepare the composition. In an embodiment, the nanoparticle formulation of talazoparib (NpTLZ) comprises PLGA with a 1:1 ratio of lactic acid and glycolic acid and was prepared using a single emulsion technique.

Size of the nanoparticles is manipulated to achieve the desired permeability and retention effects during cancer therapy. In certain embodiments, the size of the nanoparticles is less than 500 nm. In certain embodiments, the size of the blank nanoparticle, containing PLGA with a 1:1 ratio of lactic acid and glycolic acid, ranges from 138 nanometers (nm) to 230 nm. In certain embodiments, the size of the TLZ nanoparticle, containing PLGA with a 1:1 ratio of lactic acid and glycolic acid, ranges from 151 nm to 275 nm. Table 1 presents the physiochemical characteristics of the blank nanoparticle formulation and the talazoparib-containing nanoparticle formulation. Polydispersity Index (PD) defined as the standard deviation of the particle diameter distribution divided by the mean particle diameter was calculated for the two nanoparticle formulations. PD values closer to 0 represent uniformly sized particles. In an embodiment, the PD value of the blank nanoparticle was 0.15, whereas the PD value of the TLZ nanoparticle was 0.2. The nanoparticles are measured by dynamic light scattering method using an automatic particle sizer designed for use with either concentrated suspensions of small particles or solutions of macromolecules. One such sizer is the 90Plus PALS machine from Brookhaven Instruments Corporation, Brookhaven Corporate Park, 750 Blue Point Road, Holtsville, N.Y. 11742, U.S.A. There was no significant difference in the PD values of unloaded and TLZ loaded nanoparticles.

TABLE 1

| | | | | | Amount of |
|---|---|---|---|---|---|
| | | | Zeta | Loading | talazoparib |
| Particle | Size | | Potential | Efficiency | per mg of NP |
| Group | (nm) | PD | (mV) | (%) | (μg) |
| Unloaded | 184 ± 46 | 0.15 | −29 ± 9 | N/A | N/A |
| nanoparticles | | | | | |
| Talazoparib | 213 ± 62 | 0.2 | −27 ± 14 | 44 | 75 |
| loaded | | | | | |
| nanoparticles | | | | | |

Disclosed here are methods of parenteral administration of the formulation with talazoparib-containing nanoparticles and temozolomide (NpTLZ+TMZ). The parenteral administration of the NpTLZ+TMZ formulation accumulates in the tumor based on the enhanced permeability and retention effect. Embodiments include doses and dose regimens that achieve complete disappearance of tumors. For example, when this formulation was tested in animals, a regimen led to complete tumor disappearance and there was no regrowth in most animals for at least as long as twelve weeks. The regimen used here was a once-weekly dose of 5.6 mg/kg of NpTLZ for three weeks administered intravenously combined with a five times-daily dose of 40 mg/kg of TMZ administered orally. These results have demonstrated significant improvement compared to administration of temozolomide with TLZ (not presented as nanoparticles). In the example of studies using TMZ and TLZ, the tumors started re-growing between five to seven weeks. And this set of animals were administered a higher dose of TMZ (40 mg/kg vs 30 mg/kg with the same schedule) compared to the free-TLZ combination. This dose used composed 60% of maximum tolerated dose (MTD). Importantly, these studies demonstrate the feasibility of a once weekly dose, which provides clinical benefits for pediatric patients.

In a Pediatric Preclinical Testing Program study, the maximum tolerated dose for temozolomide combined with free-talazoparib (0.1 mg/kg PO BID daily x 5) was 30 mg/kg. The efficacy testing of the combination of NpTLZ with temozolomide showed maintained complete response (MCR) in TC-71 xenograft at much higher temozolomide dose (NpTLZ at 5.6 mg/kg once/week×3 IV; temozolomide at 40 mg/kg BID×5 PO). These data show that in combination with NpTLZ, the dose of temozolomide could be increased to about >75% of the maximum tolerated dose. This leads to re-sensitization of tumors intrinsically resistant to the combination of free-talazoparib and temozolomide. This strongly indicates that higher doses of temozolomide may be tolerated in combination with NpTLZ. The use of nano-formulated talazoparib may overcome intrinsic resistance to cytotoxic alkylating agents, due to higher drug accumulation in tumor tissue. As there is decreased normal tissue distribution of NpTLZ, higher doses of temozolomide are tolerated. The parenteral administration of the NpTLZ and the NpTLZ+TMZ formulations have advantages for young patients with respect to compliance as these patients fail to strictly adhere to oral administration regimens.

### EXAMPLES

The following Examples are set forth to aid in the understanding of the embodiments of the invention, and are not intended and should not be construed to limit in any way the embodiments set forth in the claims which follow thereafter.

### Example 1

TLZ loaded PLGA nanoparticles were made using the single emulsion method. About 5 mg of TLZ (MedChemExpress, N.J., USA), and 100 mg of poly lactic-co-glycolic acid (PLGA with L/G ratio of 50:50) in a 3 ml-chloroform solution was added dropwise to a 5% w/v polyvinyl alcohol solution and sonicated for 10 minutes with alternating intervals of 1.5 minutes of sonication and 1 minute of break. The mixture was then subject to overnight stirring to evaporate the solvents. The nanoparticle suspension was subject to centrifugation at 15,000 rpm for 30 minutes. The TLZ-loaded PLGA nanoparticles were retrieved, washed, and lyophilized. In certain embodiments, the nanoparticles can be dialyzed against distilled water and collected using ultracentrifugation and lyophilization.

The antitumor activity of the blank nanoparticles, nanoparticle formulations of talazoparib, temozolomide alone, and nanoparticle formulations of talazoparib in combination with temozolomide were evaluated in sensitive Ewing sarcoma models.

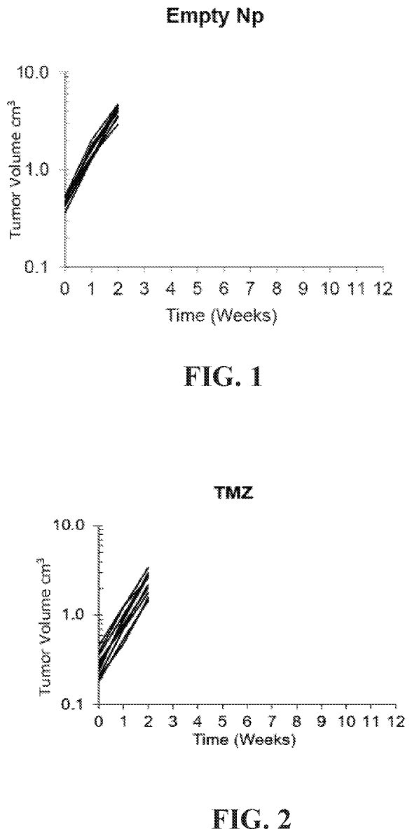

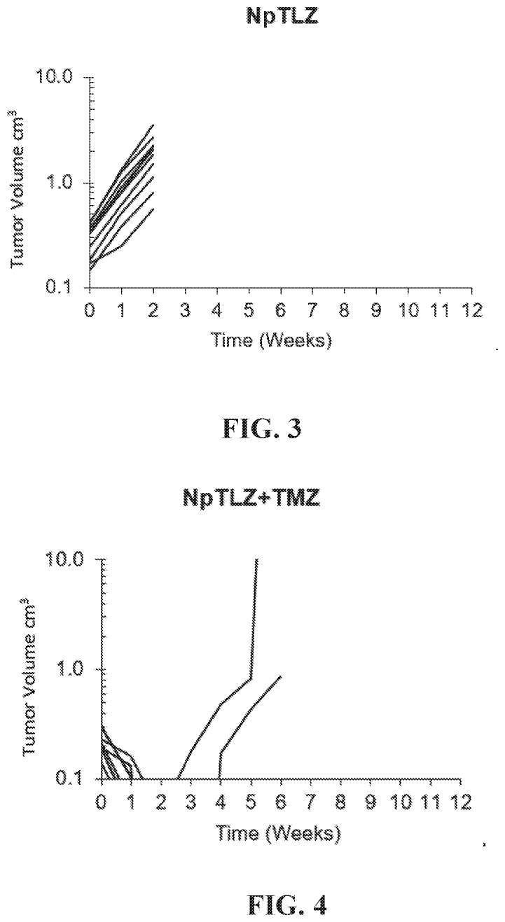

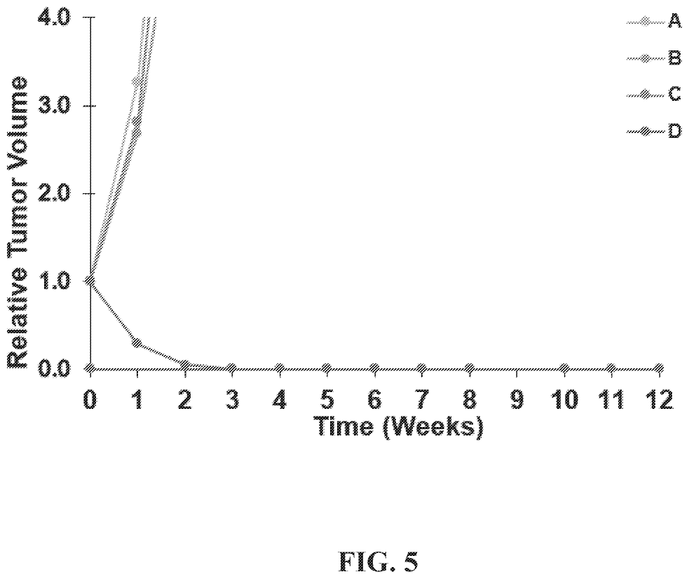

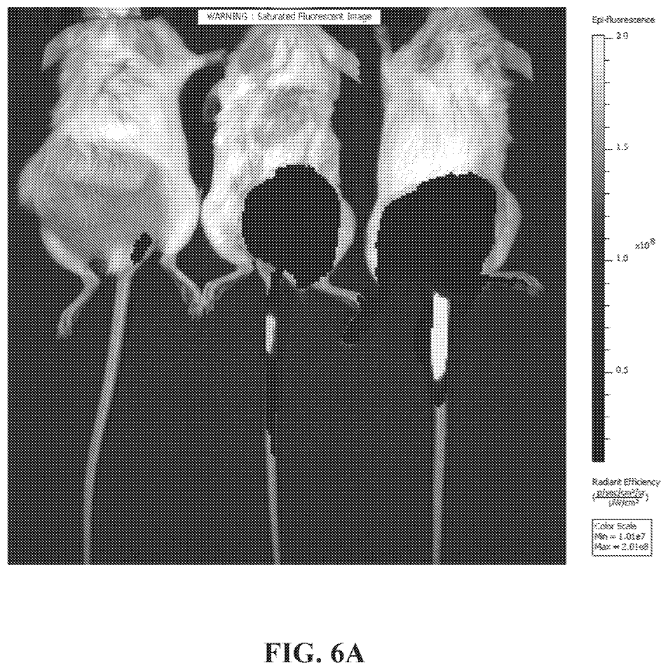

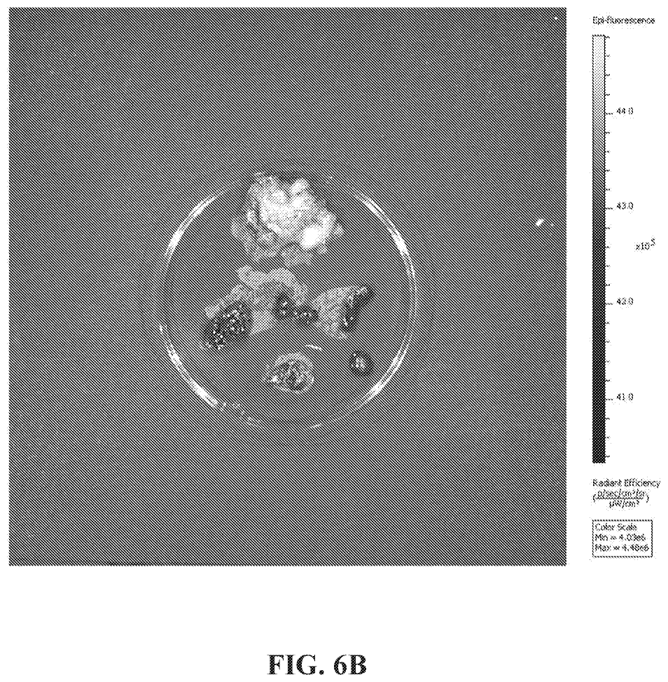

Four sets of mice bearing naïve TC-71 Ewing sarcoma xenografts were treated with either blank nanoparticles, nanoparticle formulations of talazoparib, temozolomide alone, or nanoparticle formulations of talazoparib in combination with temozolomide. In these efficacy testing models, 10 mice per group were used. CB-17 SCID mice of 6-8 weeks old were transplanted with tumors. The length and height of the tumor were measured to calculate tumor volume. When tumors reached at least 100 mm³ in size, the mice were subject to the various treatments. Parenteral administration (IV) was used to achieve systemic delivery of nanoparticles to the tumor site. TMZ was administered orally. The initial dosage regimen was a daily dose of 1 mg/kg of NpTLZ for five days. Then, the doses were combined to one single dose administered once weekly. Prolonged treatment of weeks allowed for a longer major clinical response (at least, for up to 12 weeks). FIG. 1 is a graphical representation of the changes in volume of the tumors over time in animals administered with blank nanoparticles. The control group was administered empty nanoparticles one time per week for three weeks. According to the applicable animal protocol, measurements were stopped when tumors reach 4× of initial size and animals were euthanized. FIG. 2 is a graphical representation of the changes in volume of the tumors over time in animals that were administered temozolomide alone. This group was administered 40 milligrams of temozolomide per kilogram weight of the mice twice a day for five days. Overall, this group had similar to the empty nanoparticles group response. FIG. 3 is a graphical representation of the changes in volume of the tumors over time in animals administered with nanoparticle formulations of talazoparib. This group was administered 5.6 milligrams of nanoparticles per kilogram weight of the mice once per week for three weeks. Tumors were growing but with lower rate compared to empty nanoparticles group. FIG. 4 is a graphical representation of the changes in volume of the tumors over time in animals that were administered with temozolomide and the nanoparticle formulation of talazoparib. This group was administered 5.6 milligrams of nanoparticles and 40 milligrams of temozolomide per kilogram weight of the mice once per week for 3 weeks. The tumors didn't increase in volume, and in fact, they completely disappeared. All tumors started reducing in size from the beginning of the treatment. By week 5, they completely disappeared. The measurements of tumor volume were done for 12 weeks. FIG. 5 is a graphical representation of the relative tumor volume (RTV) over time in animals that were administered with blank nanoparticles (A), nanoparticle formulations of talazoparib (B), temozolomide alone (C), and nanoparticle formulations of talazoparib in combination with temozolomide (D), corresponding to FIGS. 1-4 in the present application.

### Example 2

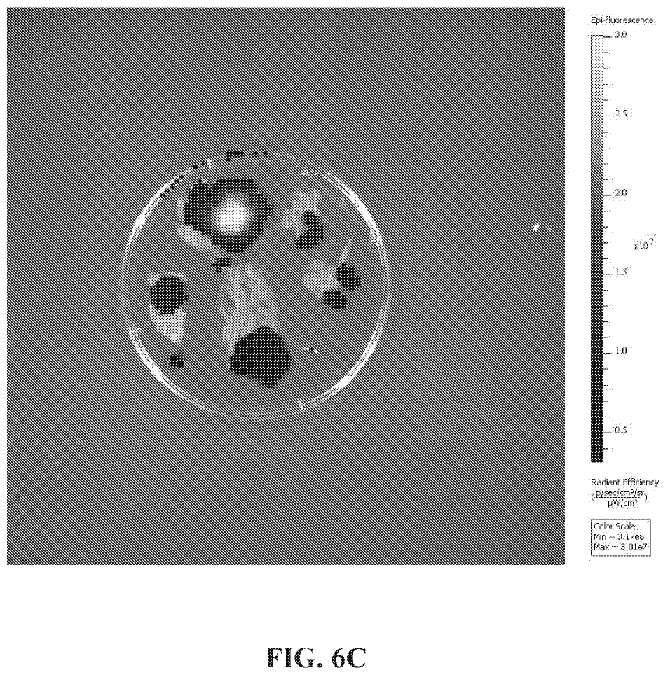

Mice were injected intravenously through tail vein with 2 mg/ml of fluorescent PLGA-Alexfluor790 nanoparticles (Phosphorex, Inc.). Imaging was acquired at 72 hours using the IVIS Xenogen Imaging System (Perkin Elmer, excitation/emission. FIGS. 6A-6C are images showing enhanced permeability and retention effect of the nanoparticle formulations of talazoparib. FIG. 6A is a photographic image of the three mice that have been treated with either no dyed injection (left mouse), or with NpTLZ (center mouse and right mouse). FIG. 6B is an image of isolated organs from the control mouse that has not been treated with a nanoparticle formulation (1-tumor, 2-kidney, 3-liver, 4-lung, 5-spleen, and 6-heart). There is no accumulation of the dye in the tumor. FIG. 6C is an image of isolated organs from the mouse that has been treated with the nanoparticle formulation (1-tumor, 2-kidney, 3-liver, 4-lung, 5-spleen, and 6-heart). The dye accumulates in the tumor when administered in the form of nanoparticles. A color scale has been provided to translate the intensity of color to the amount of accumulation of the nanoparticles. The intensity increases from yellow to brown color with the increase in the amount of accumulation of the nanoparticles.

### Example 3

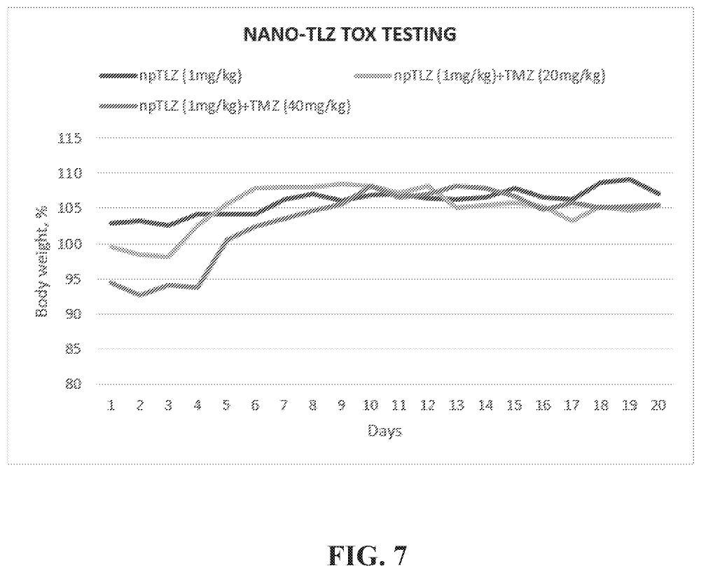

The toxicity of the nanoparticle formulations of talazoparib in combination with temozolomide and the nanoparticle formulations of talazoparib alone were tested in sensitive Ewing sarcoma mice models. CB-17 SCID mice of 6-8 weeks old were transplanted with tumors. When tumors reached at least 100 mm³ in size, treatments were administered. Parenteral administration (IV) was used to achieve systemic delivery of NpTLZ to tumor site. TMZ was administered orally. Tumors were not harvested. And, after the last measurements, the mice were euthanized. FIG. 7 is a graphical representation of the changes in the body weight of animals who were administered nanoparticle formulations of talazoparib and nanoparticle formulations of talazoparib in combination with two different doses of temozolomide. The weight loss in animals administered with the higher dose of temozolomide in combination with the nanoparticle formulations of talazoparib was less than 7% as compared to animals administered with only nanoparticle formulations of talazoparib of body weight. Surprisingly, increased dose of temozolomide did not adversely affect the body weight of the animals when administered along with separate doses of nanoparticle formulations of talazoparib.

### Example 4

The release profile of TLZ was evaluated from nanoparticles made from different molecular weight PLGAs: a high molecular weight PLGA with a molecular weight ranging from 55-65 kiloDaltons, and a low molecular weight PLGA with a molecular weight ranging from 1,000-5,000 Daltons, both containing a 1:1 ratio of lactic acid and glycolic acid and available from Akina, Inc., West Lafayette, Ind.

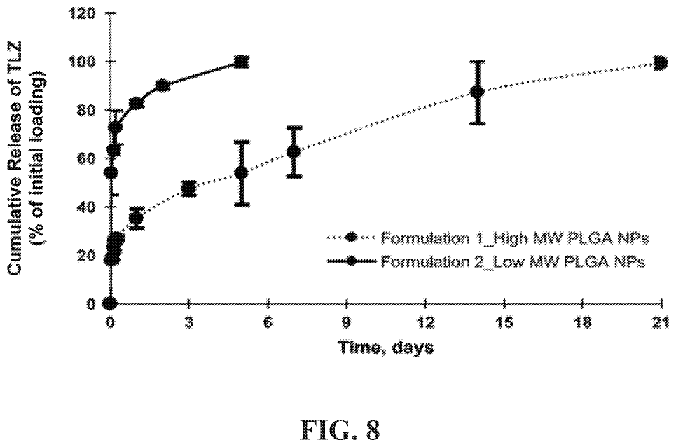

The TLZ nanoparticles were suspended in phosphate buffered saline (PBS) and sealed in dialysis bags with a molecular weight cut-off size to permit release of TLZ but not the nanoparticles. The nanoparticles were dialyzed against PBS with gentle agitation. At predetermined time points, the dialysates were collected and analyzed for the concentration of TLZ. The drug release kinetics were expressed as the percentage of the amount of TLZ released cumulatively from the total amount of TLZ loaded in the nanoparticles. FIG. 8 is a graphical representation of the release profile of TLZ from nanoparticles of two different molecular weights. The high molecular weight PLGA NPs facilitated a slower and sustained release of the TLZ. Depending on the treatment regimen, suitable molecular weight nanoparticles can be utilized to deliver the PARP1 inhibitors. Those skilled in the art will recognize, or be able to ascertain using no more than routine experimentation, many equivalents to the specific embodiments of the invention described herein. Such equivalents are intended to be encompassed by the following claims.

What is claimed is:

1. A method of treatment of cancer in a subject in need thereof, the method comprising:
administering a therapeutically effective amount of a cytotoxic alkylating agent and a nanoparticle formulation containing an inhibitor of poly (ADP-ribose) polymerase 1 or pharmaceutical acceptable derivatives thereof.

2. The method of claim 1, wherein the therapeutically effective amount of the cytotoxic alkylating agent is administered orally and the nanoparticle formulation containing the inhibitor of poly (ADP-ribose) polymerase 1 is administered through a parenteral route.

3. The method of claim 1, wherein the therapeutically effective amount of the cytotoxic alkylating agent and the nanoparticle formulation containing the inhibitor of poly (ADP-ribose) polymerase 1 is administered to treat Ewing's sarcoma.

4. The method of claim 1, wherein the therapeutically effective amount of the cytotoxic alkylating agent and the nanoparticle formulation containing the inhibitor of poly (ADP-ribose) polymerase 1 is administered to treat breast cancer.

5. The method of claim 1, wherein the cytotoxic alkylating agent is one or more of a nitrogen mustard, a nitrosurea, an ethylenimine, an alkylsulfonate, a hydrazine, a triazines, or a metal salt.

6. The method of claim 1, wherein the cytotoxic alkylating agent is one or more of cyclophosphamide, melphalan, ifosfamide, or irinotecan.

7. The method of claim 1, wherein the cytotoxic alkylating agent is an imidazotetrazine.

8. The method of claim 1, wherein the cytotoxic alkylating agent is temozolomide.

9. The method of claim 1, wherein the inhibitor of poly (ADP-ribose) polymerase 1 is a benzimidazole-based compound.

10. The method of claim 1, wherein the inhibitor of poly (ADP-ribose) polymerase 1 is one or more of niraparib, rucaparib, olaparib, and veliparib.

11. The method of claim 1, wherein the inhibitor of poly (ADP-ribose) polymerase 1 is talazoparib.

12. The method of claim 1, wherein the nanoparticle formulation comprises poly lactic-co-glycolic acid.

13. A pharmaceutical composition for treatment of cancer comprising a nanoparticle formulation containing an inhibitor of poly (ADP-ribose) polymerase 1 or a pharmaceutical acceptable derivative thereof.

14. The pharmaceutical composition of claim 13, wherein the inhibitor of poly (ADP-ribose) polymerase 1 is a benzimidazole-based compound.

15. The pharmaceutical composition of claim 13, wherein the inhibitor of poly (ADP-ribose) polymerase 1 is one or more of niraparib, rucaparib, olaparib, and veliparib.

16. The pharmaceutical composition of claim 13, wherein the inhibitor of poly (ADP-ribose) polymerase 1 is talazoparib.

17. The pharmaceutical composition of claim 13, wherein the nanoparticle formulation comprises poly lactic-co-glycolic acid.

18. The pharmaceutical composition of claim 17, wherein the poly lactic-co-glycolic acid contains a 1:1 ratio of lactic acid and glycolic acid.

19. The pharmaceutical composition of claim 17, wherein the nanoparticle formulation comprises high molecular weight poly lactic-co-glycolic acid.

20. The pharmaceutical composition of claim 13, wherein the nanoparticle formulation is prepared for parenteral administration.
